(19) United States
(12) Patent Application Publication
(10) Pub. No.: US 2018/0249566 A1
(43) Pub. Date: Aug. 30, 2018

(54) X-RAY SOURCE

(71) Applicant: KONINKLIJKE PHILIPS N.V., EINDHOVEN (NL)

(72) Inventors: Roland PROKSA, Neu Wulmstorf (DE); Lester Donald MILLER, HUDSON, OH (US)

(21) Appl. No.: 15/753,698

(22) PCT Filed: Aug. 24, 2016
(86) PCT No.: PCT/EP2016/070002

§ 371 Date: Feb. 20, 2018

Related U.S. Application Data
(60) Provisional application No. 62/210,604, filed on Aug. 27, 2015.

(51) Int. Cl. H05G 1/20; A61B 6/03; A61B 6/00; H05G 1/08; H05G 1/50

(52) U.S. Cl. CPC H05G 1/20; A61B 6/032; A61B 6/4035; H05G 1/085; H05G 1/50; A61B 6/405

(57) ABSTRACT

The invention relates to an X-ray source (2) for an imaging device comprising at least three electrodes; a power supply configured to provide a primary gap voltage between a first (13) and a second (12) electrode among said at least three electrodes, said primary gap voltage having an AC component, causing a transport of electrons from the first electrode toward the second electrode; and a controller configured to supply a variable potential on a third electrode (14) among said at least three electrodes, wherein the X-ray source is configured to generate an X-ray beam with an energy spectrum based on the voltage difference between the first electrode and the second electrode, and wherein the controller is configured to set the variable potential on the third electrode to a value causing at least a partial blocking of said transport of electrons, whenever a predetermined condition is met.

### FIELD OF THE INVENTION

The present invention relates to an X-ray source, an imaging device and a method of controlling an energy level of an X-ray beam in an X-ray source. It can be applied to Computed Tomography devices and including spectral Computed Tomography devices.

### BACKGROUND OF THE INVENTION

When imaging an object or a patient with X-ray, the quality of the final image mainly depends on the energy of the X-ray used.

As a matter of fact, the more photons are able to reach the detector through the object or the patient to be imaged, the lower the image noise. Depending on certain parameters of the object or patient, for instance its thickness, a different amount of energy will be needed to allow the photons to get through. However, low energy photons which are typically stronger absorbed carry more important contrast information. In thick objects the loss of low energy photons may become too high (beam hardening) and may require more high energy photons in the spectrum to reach an acceptable image noise level. The ideal X-ray energetic profile depends of course on the object or patient to be imaged (hereinafter, the terms “X-Ray spectrum” will be used indifferently to designate an X-Ray energetic profile since each wavelength corresponds to an energy value).

### SUMMARY OF THE INVENTION

It is an object of the invention to provide an X-ray source and method enabling to shape the X-ray beam used, either statically or dynamically.

According to a first aspect of the invention this object is achieved by an X-ray source for an imaging device comprising:
at least three electrodes,
a power supply configured to provide a primary gap voltage between a first and a second electrode among said at least three electrodes, said primary gap voltage having an AC component, causing a transport of electrons from the first electrode toward the second electrode, and
a controller configured to supply a variable potential on a third electrode among said at least three electrodes,

wherein the X-ray source is configured to generate an X-ray beam with an energy spectrum based on the voltage difference between the first electrode and the second electrode,

and wherein the controller is configured to set the variable potential on the third electrode to a value causing at least a partial blocking of said transport of electrons, whenever a predetermined condition is met.

The first electrode is usually known as “cathode” and the second electrode is usually known as “anode”. The third electrode is usually referred to as “grid”, although it does not necessarily have a grid shape. The third electrode can actually be grid-shaped, but it may have any suitable shape which does not completely block the transport of electrons between the first and the second electrode. The third electrode is preferably located between the first and the second electrode but it may also be located elsewhere provided the actual layout of the X-ray source does not prevent it to fulfill its function.

The X-Ray source according to the invention usually has three electrodes, but it also may have a larger amount of electrodes. In particular, any of the first, second and/or third electrode may be replaced by several electrodes cooperating together to fulfill the same function. It can be especially useful in the case of the “grid” as it can allow more complex shapes with an increased efficiency. The X-Ray source may comprise other electrodes, such as a base electrode or a gate.

The first and the second electrodes may be made of the same material or from different materials. They are preferably made from a metallic material, for instance a material chosen from tungsten, molybdenum or copper. The material can be a complex assembly or a composition, for instance a complex assembly or a composition designed to resist high temperatures, such as a tungsten-rhenium target on a molybdenum core.

The first and the second electrodes may have any shape. Preferably, the second electrode has a shape with a circular symmetry and is rotatable.

The transport of electrons between the first and the second electrodes, caused by the primary gap voltage, is a current. Its value depends both on the primary gap voltage, on the electron emission rate of the cathode and on the material which is filling the space between the first and the second electrodes. Preferably, all the electrodes are located in an enclosure filled with a suitable material, for instance very low pressure inert gas which may even be considered to be vacuum. This allows for optimal transportations of the electrons between the electrodes. The electrons transported from the first electrode would eventually collide with the second electrode, transferring part of their energy to other electrons from its metallic structure, and converting a residual part of their energy into X-ray radiation.

An X-ray detector, located outside of the X-ray generator, detects the photons generated by said collision on a pre-determined period based on the detector technology chosen. In the context of the invention, the detector may be of any suitable type known to the skilled person.

The output spectrum depends on the values of the primary gap voltage: the output spectrum is kVp dependent.

If the third electrode is set to an appropriate value, for instance a very negative value, all the electrons from the cathode cannot pass the third electrode, thus effectively stopping the X-ray emission.

In a preferred embodiment, the third electrode is designed so that electrons do not hit the third electrode. If the potential of the third electrode is negative, electrons are blocked, whereas if it is positive the total electric field guides the electrons toward the anode.

By resetting the third electrode to its initial value, the transport of electrons resumes toward the second electrode and the X-ray radiation is emitted again from the source.

It is important to note that the initial value of the potential applied to the third electrode, the “grid”, has to be chosen such that any unwanted leakage current towards it is avoided. Preferably, the initial value of the potential applied to the third electrode is higher than the potential applied to the first electrode, the “cathode”.

The above layout thus allows a very fast switching on and off of the X-ray source.

In the main embodiment of the invention, the primary gap voltage has an AC component. This generates a continuously changing output spectrum. The frequency of the AC component frequency is chosen such that the integration period of the X-Ray detector is one or more periods of the AC component. Because of that, the effective spectrum detected by the detector is an average on the spectrum.

Using the previously described fast-switching system, it is possible to selectively allow electrons transportation when a predetermined condition is met, preferably when the primary gap voltage has pre-determined values of interests. This allows to control which kVp contributes to the average spectrum, and thus shapes the X-Ray beam.

Preferably, the variable potential on the third electrode is set to the value causing at least partial blocking of said transport of electrons whenever the primary gap voltage lies between a minimum extinction value and a maximum extinction value. This allows for effective kVp control: the kVp comprised between those extinction values can thus not contribute to the emitted spectrum.

The minimum extinction value can be comprised between 30 kVp and 80 kVp whereas the maximum extinction value can be comprised between 80 kVp and 160 kVp. However, in some applications, for instance in case a DC component is provided, the extinction values can exceed these boundaries.

Any kVp value which is not comprised in a certain interval may be blocked by setting the variable potential on the third electrode to a value causing at least a partial blocking of said transport of electrons whenever the primary gap voltage does not lie between an appropriate minimum and maximum trigger value.

Preferably, the primary gap voltage has a DC component. The DC component allows to strengthen the flux of electrons and to avoid any transport of electrons from the second electrode to the first electrode. The DC component also allows to choose among which range of values the kVp of the X-ray spectrum will be selected. Preferably, said DC component lies between 80 kilovolts and 150 kilovolts, more preferably between 90 kilovolts and 120 kilovolts, and even more preferably of about 100 kilovolts.

The variable potential on the third electrode may be set to a value causing at least a partial blocking of said transport of electrons at regular intervals, said intervals corresponding to a given first frequency. This allows to apply any common periodic voltage between the third electrode and the ground, for instance a sinusoidal voltage. Such voltage is easy to tune using common signal processing techniques, and may be effectively shaped depending on the primary gap voltage. In particular, said first frequency may be set to match the frequency of the AC component of the primary gap voltage in order to make sure it is always the same kVp which contributes to the output X-ray spectrum for each consecutive period.

Preferably, the variable potential on the third electrode is a crenel voltage. Although such a voltage includes very high frequency components which might be troublesome, it allows to make a clear cut between the time when the electrons from the first electrode are transported towards the second electrode, the “on position” of the switch corresponding to the high value of the crenel voltage, and the time when the electrons cannot be transported toward the second electrode, the “off position” corresponding to the low value of the crenel voltage. In some cases, it might be preferable to use a voltage without any high frequency components.

The AC component of the primary gap voltage can have a frequency comprised between 10 Hz. and 20 kHz , preferably close to the readout frequency of the detector. The frequency of the AC component is preferably high enough so that the detector averages the output spectrum over one or more periods of the AC component.

The X-ray source may further comprise a transformer. Specifically, the transformer may be configured to adapt an impedance of the at least three electrodes to the tube in order to obtain a resonating circuit. Such a resonating circuit can be of interest in terms of energy saving, and decreases the amount of heat produced by the system.

The invention also relates to an imaging device comprising an X-Ray source according to the present invention.

The imaging device according to the invention is preferably a Computed Tomography device, including for instance a Spectral Computed Tomography device, but any other medical imaging device comprising an X-Ray source benefits from the invention.

According to another aspect of the invention a method of controlling an energy level of an X-ray beam in an X-Ray source comprises:
generating a primary gap voltage causing a transport of electrons from a first electrode toward a second electrode, the electrons hitting said second electrode generating an X-Ray beam,
setting a potential on a third electrode to a value causing at least a partial blocking of said transport of electrons whenever a predetermined condition is met.

Such method is preferably implemented using the above described device, but can also be implemented using any other suitable layout.

### BRIEF DESCRIPTION OF THE FIGURES

The invention shall be better understood by reading the following detailed description of an embodiment of the invention and by examining the annexed drawing, on which:

FIG. 1 is schematically represents a general layout of a device according to the invention,

FIG. 2 represents instant X-Ray spectra corresponding to different kVp-values applied to a standard X-Ray source,

FIG. 3 is a diagram showing the different voltages applied to the electrodes of a device according to the invention over time, and the resulting X-ray.

In order to implement the main embodiment of the invention, a grid switch 11 is set up.

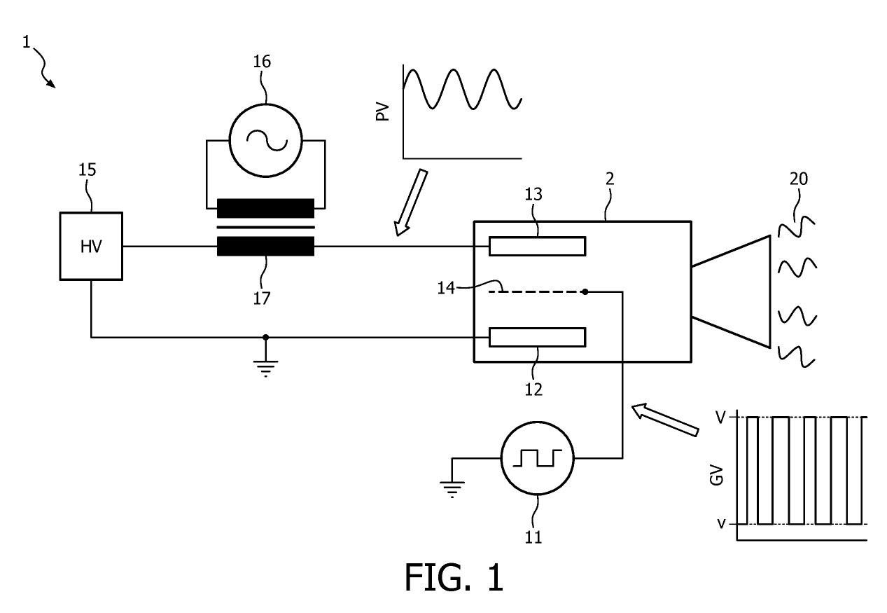

FIG. 1 represents a device 1 according to the invention. An X-ray tube 2, comprises a first electrode, the cathode 13, and another electrode, the anode 12.

X-ray are produced in a usual way by sending high energy electrons from the cathode 13, toward the anode 12. Part of the energy of the electrons is absorbed by the anode 12, and a small part of it is restituted by emitting X-ray radiation 20. In order to induce the transportation of electrons from the cathode 13 to the anode 12, a primary gap voltage PV is applied between the cathode 13 and the anode 12.

The resulting emitted X-ray spectrum depends on the energy of the transported electrons, and thus on said primary gap voltage PV. In order to have different energetic contributions to the output spectrum, the primary gap voltage PV has both a high-voltage DC component, or offset, produced by a high voltage generator 15, and an AC component produced by an AC generator 16. The AC component and the DC component are summed up together by a transformer 17. The generator 16, the transformer 17 and tube can be designed as a resonating circuit.

Between the anode 12 and the cathode 13, a grid-shaped electrode 14 is inserted. The grid-shaped electrode 14 is part of a grid switch system 11, which further comprises a controller which enables to apply a certain grid potential GV, a crenel voltage, to the electrode 14. Said grid potential GV allows to stop the X-ray emission 20 very quickly when desired.

The terms grid switch refer to a X-ray tube internal layout known in the art allowing for a very fast extinction of the resulting X-ray beam.

Due to capacitive effects, the X-ray emission does not stop instantly when the primary gap voltage PV is set to zero. The grid switch allows to solve this issue.

The grid switch 11 has two positions: an on position and an off position. When the switch is on, the switch interferes as little as possible with the electrons travelling from the cathode 13 toward the anode 12. Therefore, the potential of the electrode 14 is set to a highly positive value V. When the switch is off, the switch impedes the electrons emitted by the cathode 13 to reach the anode 12. Therefore, the potential is set to a highly negative value v.

The transition between the on and off positions is controlled by a controller, not represented on the drawing, which sets the potential GV on the electrode 14 accordingly. The transition may be made in any way, but the most convenient way to switch between two constant voltage values is to use a voltage with a crenel-shaped voltage curve.

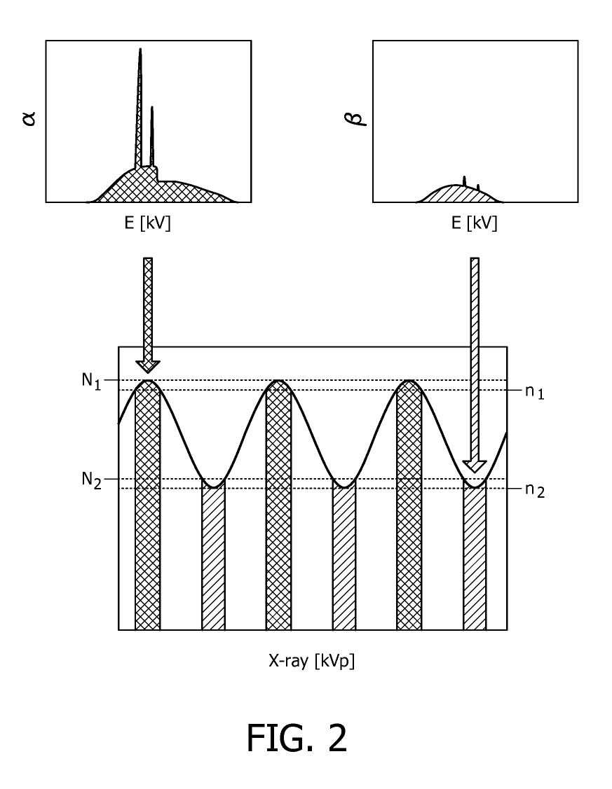

The grid potential GV is chosen in order to allow only certain values of the primary gap voltage PV to contribute to the average output X-ray spectrum. FIG. 2 illustrates two possible grid potentials GV and the resulting output X-ray spectra. In a first embodiment, corresponding to the plain gray area of the diagram, the grid switch is set to its on position when the primary gap voltage PV is comprised between a minimum value n1 and a maximum value N1; n1 and N1 defining an interval comprising the maximum values of the primary gap voltage PV. When the primary gap voltage PV does not lie between these values, the grid switch is set to its off position. Because of this configuration, the resulting X-ray spectrum, the α diagram plotted on the left-high corner of FIG. 2, has energetic contributions comprised only between n1 and N1: it allows to have a spectrum with a high energy tail, which may be useful to image thick patients for instance. In a second embodiment, corresponding to the hatched area of the diagram, the grid switch is set to its on position when the primary gap voltage PV is comprised between a minimum value n2 and a maximum value N2; n2 and N2 defining an interval comprising the minimum values of the primary gap voltage PV. When the primary gap voltage PV is not comprised between these values, the grid switch is set to its off position. Because of this configuration, the resulting X-ray spectrum, the β diagram plotted on the right-high corner of FIG. 2, has energetic contributions comprised only between n2 and N2, which allows for low kVp spectrum which allows for better contrast images.

Both these embodiments imply that the grid potential GV is a periodic potential with a frequency identical to the primary gap voltage PV, in order to always cut the same values of the voltage.

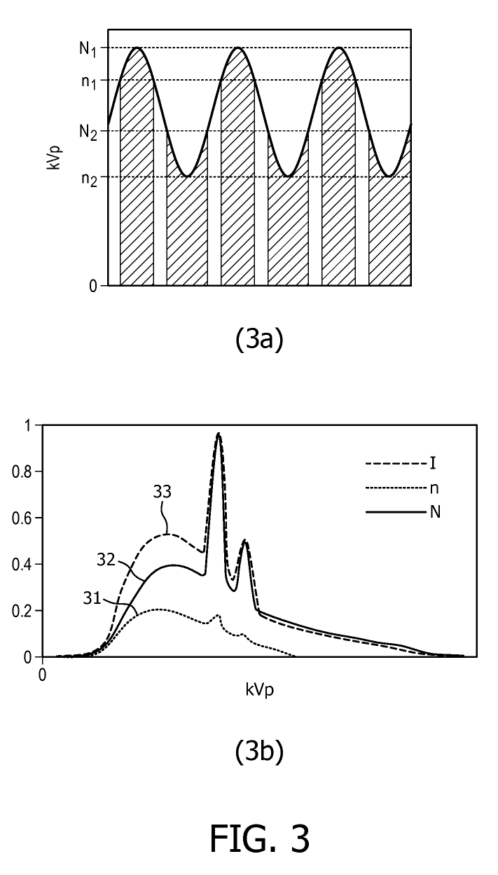

The invention also allows to combine several energy ranges, as illustrated in FIG. 3. In FIG. 3, the grid switch is set to its on position each time the primary gap voltage is either between n1 and N1 or between n2 and N2, and set to its off position otherwise. This allows to shape the output spectrum. FIG. 3b represents three different output X-ray spectra. The first spectrum, plotted with a grey thin line 31, corresponds to the X-ray spectrum detected if the only energetic contributions to the spectrum are low-kVp, such as the one comprised between n2 and N2. The second spectrum, plotted with a plain thick line 32, corresponds to the X-ray spectrum detected if the only energetic contributions to the spectrum are high-kVp, such as the one comprised between n1 and N1. Eventually, the third spectrum, plotted with a dotted line 33, corresponds to a spectrum with both contributions. The latter has a high energy tail which allows to image effectively thicker patients but also have an increased low-energy part, which allows for better contrasts.

By adapting the combination, the one skilled in the art obtains a lot of freedom to many ways of increasing the contributions according to the needs, for instance according to the physiology of the patient who is to be imaged.

The one skilled in the art could also use a different grid potential GV. As a matter of fact, a crenel-shaped voltage only allows two positions of the grid switch system, and thus only permits to shut down certain radiations. By using a shaped voltage, it is possible to give a weight to each value and thus control the beam energy more finely.

Controlling the beam energy allows to minimize the dose of X-ray radiation effectively received by the patient imaged.

While the invention has been illustrated and described in detail in the drawings and foregoing description, such illustration and description are to be considered illustrative or exemplary and not restrictive; the invention is not limited to the discussed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

1. X-ray source for an imaging device comprising:
at least three electrodes,
a power supply configured to provide a primary gap voltage between a first and a second electrode among said at least three electrodes, said primary gap voltage having an AC component and having a DC component, causing a transport of electrons from the first electrode toward the second electrode and part of the energy of the electrons is absorbed by the second electrode, and a small part of it is restituted by emitting X-ray radiation, and
a controller configured to supply a variable potential on a third electrode among said at least three electrodes,
wherein the X-ray source is configured to generate an X-ray beam with an energy spectrum based on the voltage difference between the first electrode and the second electrode, and
wherein the controller is configured to set the variable potential on the third electrode being set to the value causing at least a partial blocking of said transport of electrons, impeding the electrons emitted by the first electrode to reach the second electrode, whenever the primary gap voltage is comprised between a minimum extinction value (N2) and a maximum extinction value (n1).

2. (canceled)

3. X-Ray source according to claim 1, the minimum extinction value being comprised between 30 kVp and 80 kVp .

4. X-Ray source according to claim 3, the maximum extinction value being comprised between 80 kVp and 160 kVp.

5. (canceled)

6. X-Ray source according to claim 1, said offset DC component being comprised between 80 kilovolts and 150 kilovolts, preferably between 90 kilovolts and 120 kilovolts, more preferably of 100 kilovolts.

7. X-Ray source according to claim 6, the variable potential on the third electrode being set to the value causing at least the partial blocking of said transport of electrons at regular intervals, said intervals corresponding to a given first frequency.

8. X-Ray source according to claim 7 said first frequency matching the frequency of the AC component of the primary gap voltage.

9. X-Ray source according to claim 6, the variable potential on the third electrode having a crenel-shaped voltage curve.

10. X-Ray source according to claim 6, the AC component of the primary gap voltage having a frequency comprised between 10 Hz and 20 kHz , preferably close to the readout frequency of the detector.

11. X-Ray source according to claim 10, the X-ray source further comprising a transformer.

12. X-Ray source according to claim 11, the transformer being configured to adapt an impedance of the at least three electrodes to the tube in order to obtain a resonating circuit.

13. Imaging device comprising an X-Ray source according to claim 1.

14. Imaging device according to the claim 13, being a Computed Tomography device.

15. Method of controlling an energy level of an X-ray beam in an X-ray source comprising:
generating a primary gap voltage causing a transport of electrons from a first electrode toward a second electrode, the electrons hitting said second electrode generating an X-Ray beam,
setting a potential on a third electrode being set to the value causing at least a partial blocking of said transport of electrons, impeding the electrons emitted by the first electrode to reach the second electrode, whenever the primary gap voltage is comprised between a minimum extinction value (N2) and a maximum extinction value (n1).

16. X-Ray source according to claim 1, wherein the controller is configured to set the variable potential on the third electrode being set to the value causing said transport of electrons whenever the primary gap voltage is comprised between a minimum value n1 and a maximum value N1, n1 and N1 defining an interval comprising the maximum values of the primary gap voltage PV.

17. X-Ray source according to claim 1, wherein the controller is configured to set the variable potential on the third electrode being set to the value causing said transport of electrons whenever the primary gap voltage is comprised between a minimum value n2 and a maximum value N2, n2 and N2 defining an interval comprising the minimum values of the primary gap voltage PV.
